(19) United States
(12) Patent Application Publication
(10) Pub. No.: US 2006/0217455 A1
(43) Pub. Date: Sep. 28, 2006

(54) Self hardening glass carbomer composition

(76) Inventors: Willem Frederik Van Den Bosch, Oegstgeest (NL); Raimond Nicolaas Bruno Van Duinen, Den Haag (NL)

Correspondence Address: Abelman Frayne & Schwab, 150 East, 42nd Street, New York, NY, 10017-5621, US

(21) Appl. No.: 10/559,900

(22) PCT Filed: Jun. 3, 2004
(86) PCT No.: PCT/NL04/00396

§ 371 Date: Apr. 5, 2006

Related U.S. Application Data
(60) Provisional application No. 60/475,903, filed on Jun. 5, 2003.

(30) Foreign Application Priority Data
Jun. 5, 2003 (EP) 03076770.1

(51) Int. Cl. A61L 24/00; B32B 15/02

(52) U.S. Cl. 523/113; 523/209; 428/404

(57) ABSTRACT

The present invention relates to a self hardening glass carbomer composition obtainable by treating a fluorosilicate glass powder with: (a) a poly(dialkylsiloxane) having terminal hydroxyl groups, wherein the alkyl groups contain 1 to 4 carbon atoms, (b) an aqueous acid solution, and (c) separating the treated fluorosilicate glass powder from the aqueous acid solution. The glass carbomer compositions according to the invention have for example good toughness and strength and excellent fluoride release, In addition, the glass carbomer compositions according to the invention do not show shrinkage or expansion, an essential property for providing fillings for cavities having high strength and long durability. Moreover, the glass carbomer composition according to the present invention has a lower sensitivity towards abrasion and wear, a greater stiffness, a smoother surface, a better colourfastness, a better adherence to e.g. bone tissue and a lower water sensitivity.

### FIELD OF THE INVENTION

The present invention relates to a glass carbomer cement having improved properties, a method for preparing said glass carbomer cement and the use of said glass carbomer cement in clinical and dental applications including high stress applications, e.g. tooth restoration, dentine replacement, crown core build-ups, i.e. as bone and dental cement, and industrial applications.

### BACKGROUND OF THE INVENTION

Glass ionomer cements are known in the art and are used for already a considerable period of time in clinical and dental applications, e.g. as a permanent filling material. For example, U.S. Pat. No. 4,376,835 that is incorporated by reference herein discloses a calcium aluminium fluorosilicate glass powder having a average particle size of at least 0.5 μm, a depleted calcium level at the surface of the powder particles relative to the calcium level present in the core region of the powder particles, a ratio of the Si/Ca atomic ratio at the surface of the powder particles and the Si/Ca atomic ratio in the core region of the powder particles of at least 2.0, wherein the calcium content increases asymptotically from the surface to the core region. The calcium aluminium fluorosilicate glass powder according to U.S. Pat. No. 4,376,835 has a reduced water sensitivity during and after the setting reaction and is used in a self-hardening glass ionomer cement comprising an aqueous mixture of said calcium aluminium fluorosilicate glass powder, a polycarboxylic acid and a chelating agent, wherein the polycarboxylic acid catalyses the setting or hardening reaction of the calcium aluminium fluorosilicate glass powder and the chelating agent accelerates and improve the setting or hardening reaction.

U.S. Pat. No. 5,063,257, incorporated by reference herein, addresses for example the disadvantages of some glass ionomer cements known in the art. One of the most important disadvantages of these materials is that the setting or hardening reaction is difficult to control thereby leading to cements that are embrittled at the surface and therefore have a reduced strength. U.S. Pat. No. 5,063,257 provides a solution for this problem by employing a glass ionomer cement composition comprising a fluorosilicate glass powder, a polymer of an α,β-unsaturated carboxylic acid, e.g. poly(acrylic acid), a polymerisable organic compound having unsaturated carbon-carbon bonds, a polymerisation catalyst, water, a surface active agent and a reducing agent. The setting or hardening of this composition occurs by the usual neutralisation reactions of the fluorosilicate glass powder as well as polymerisation reactions of the unsaturated groups present in the polymer of an α,β-unsaturated carboxylic acid and the polymerisable organic compound thereby giving a glass ionomer cement that is far less sensitive to water at the initial stage of setting or hardening. According to Examples 6 and 8 and 14-16, the fluorosilicate glass powder is pre-treated with an ethylenically unsaturated alkoxysilane, e.g. vinyltris(β-methoxyethoxy)silane.

U.S. Pat. No. 5,453,456, U.S. Pat. No. 5,552,485 and U.S. Pat. No. 5,670,258, all incorporated by reference, disclose fluorosilicate glass powders that are treated with an aqueous silanol treating solution and optionally an additional organic compound. These treated fluorosilicate glass powders can form cements having an improved strength. The aqueous silanol treating solutions are prepared in situ, preferably by hydrolysis of acidic ethylenically unsaturated alkoxysilanes, i.e. alkoxy silanes having preferably one or more hydrolysable alkoxy groups, one or more ethylenically unsaturated groups and one or more carboxylic groups.

Commercially available products are for example KetacMolar® from 3M ESPE and Fuji IX® from GC Corp.

However, the glass ionomer cements known from the prior art still suffer from several disadvantages. For example, the strength, stiffness and hardness of the glass ionomer cements according to the prior art is often insufficient. The surface of the known cements—when hardened—are also not very smooth with the result that, when e.g. employed as dental filing material, they are difficult to polish. Another disadvantage of the known glass ionomer cements is that the hardened cement has a rather high solubility which causes wear of the dental filling. The hardened cements also show a rather poor adherence to bone tissue. Consequently, there is still a need for improved glass ionomer cements that do suffer from these disadvantages.

In conclusion, the glass ionomer compositions according to the prior art are in particular inferior with respect to sensitivity towards abrasion and aesthetic properties. Moreover, they often show insufficient strength.

It is therefore an object of the invention to provide a glass ionomer composition (in the description reference is in general made to glass carbomer composition but both terms may be used interchangeably) that—when hardened—has improved properties with respect to the glass ionomers known in the art.

### SUMMARY OF THE INVENTION

All the methods known from the prior art that provide improved glass ionomers compositions are laborious and complicated. The present invention provides a solution for this technical problem without deteriorating effects. The glass carbomer composition according to the invention is manufactured from commonly available materials and it shows—either in the unhardened state or the hardened state—even a better performance when compared to the glass ionomer compositions known from the prior art. The glass carbomer compositions according to the invention have for example good toughness and strength and excellent fluoride release. In addition, the glass carbomer compositions according to the invention do not show shrinkage or expansion, an essential property for providing fillings for cavities having high strength and long durability.

In addition, the glass carbomer composition according to the present invention has—when hardened—in particular a higher hardness, a lower sensitivity towards abrasion and wear, a greater stiffness, a lower solubility, a smoother surface, a better colourfastness, a better adherence to e.g. bone tissue and a lower water sensitivity. Another advantage of the glass carbomer composition according to the invention is that when hardened it can be polished much easier in comparison with the known glass ionomer compositions. Further advantages of the glass carbomer composition according to the present invention is that the unhardened glass carbomer composition shows a better fluidity so that cavities are much easier to fill, a better processability and a shorter hardening time. The glass carbomer composition according to the present is also much easier to use as sealing material. All these advantages are apparent from preliminary clinical trials.

The present invention therefore relates to a self hardening glass carbomer composition obtainable by treating a fluorosilicate glass powder with:

(a) a poly(dialkylsiloxane) having terminal hydroxyl groups, wherein the alkyl groups contain 1 to 4 carbon atoms,

(b) an aqueous acid solution,

(c) separating the treated fluorosilicate glass powder from the aqueous acid solution.

### DETAILED DESCRIPTION OF THE INVENTION

The fluorosilicate glass powder particles employed in this invention are generally depleted of calcium at their surface such that the quotient of the atomic ratio Si/Ca at the surface of the powder particles and the atomic ratio Si/Ca in the core region is at least 2.0, preferably at least 3.0, and most preferably at least 4.0. The calcium content of the powder particles of the invention increases asymptotically from the surface to the core region.

The depth of the depletion zone depends on the conditions given in each individual case. However, the depletion zone preferably extends at least to a depth of about 10 nm, more preferably to at least about 20 nm, and most preferably to at least about 100 nm. These ranges are particularly suited for use of the fluorosilicate glass powders in dentistry. For other purposes, e.g., for use in bone cements, the depletion zone may also be deeper and may be 200 to 300 nm, for example.

As is known in the art, the fluorosilicate glass powders are produced by surface treatment of glass powders having a composition corresponding to the core region of the powders. Upon surface treatment the number of silicon atoms per unit volume remains substantially constant. The actual change in the absolute number of atoms per unit volume of other types of atoms is therefore obtained by forming the quotient of the relative atom proportion with the percentage silicon proportion. The quotient of the atomic ratio Si/Ca at the surface and the atomic ratio Si/Ca in the core region therefore constitutes a useful value to characterise the fluorosilicate glass powders.

The surface measurement to determine Ca depletion of the glass powders of the invention is suitably carried out by photo electron spectroscopy for chemical analysis (ESCA). This method has been described by R. S. Swingle II and W. M. Riggs in Critical Reviews in Analytical Chemistry, Vol. 5, Issue 3, pages 267 to 321, 1975 and by K. Levsen in “Chemie in unserer Zeit”, Vol. 40, pages 48 to 53, 1976. The measuring data underlying the description presented above are outlines in U.S. Pat. No. 4,376,835.

The fluorosilicate glass powders have an average particle size (weight average) of at least 0.5 μm, preferably at least 1.0 μm, and most preferably at least 3.0 μm. For dental purposes the average particle size (weight average) is 1.0 to 20.0 μm, preferably 3.0 to 15.0 μm, most preferably 3.0 to 10.0 μm. The particles have a maximum particle size of 150 μm, preferably 100 μm, especially 60 μm. For use as dental bonding cement the maximum particle size is 25 μm, preferably 20 μm. In order to achieve good mechanical properties a not excessively narrow particle size distribution is favourable, as usual, which is achieved, for example, by conventional grinding and classifying of the coarse.

The fluorosilicate glass powders are prepared from glass powders having the average composition of the core region of the powders of the invention. To this end the glass powders described, for example, in DE A 2.061.513 and in Table I are suitable. The glass powders employed as starting materials are obtained as usual by fusing the starting components together at temperatures above 950° C., quenching, and grinding. The starting components may be, for example, the compounds stated in DE A 2.061.513 in suitable quantitative ranges.

The thus obtained powders are then subjected to a surface treatment. The powders of the invention are obtainable, for example, by removal of Ca by suitable chemical agents.

For example, the starting glass powders are treated on the surface with acid, preferably at room temperature. To this end substances containing acidic groups are employed, preferably substances forming soluble calcium salts, Sparing water-solubility of the respective calcium salts may be compensated to a certain degree by a large amount of liquid per unit of powder. The reaction period varies between a few minutes and several days, depending on the strength and concentration of the acid employed.

Thus, for instance, for the preparation of the powders hydrochloric, sulfuric, nitric, acetic, propionic and perchloric acid may be used.

The acids are employed at a concentration of 0.01 to 10% by weight, preferably from 0.05 to 3% by weight.

After the respective reaction period the powders are separated from the solution and thoroughly washed to leave substantially no soluble calcium salts on the surface of the powder particles. Finally the powder is dried, preferably above 70° C., and screened to the desired particle size ranges.

The stronger the acid employed and the longer a given acid acts on the powder the longer will be the processing period after mixing with the mixing fluid.

The favourable surface character of the powders permits the use of an especially high powder/fluid ratio in the cement mix resulting in high strength values of the hardened material. The possibility of using a particularly reactive mixing fluid has the same effect. Furthermore, the processing period of a cement of the invention may be tailored to meet the user's requirements. The length of the processing period hardly influences the subsequent hardening period, so that also upon long processing periods rapid setting and early water insensitivity occurs.

The glass powders may be mixed, to form dental cements or bone cements, with the conventional aqueous polycarboxylic acid solutions as described, for example, in DE A 2.061.513, DE A 2.439.882 and DE A 2.101.889. Suitable polycarboxylic acids are polymaleic acid, polyacrylic acid and mixtures thereof, or copolymers, especially maleic acid/acrylic acid copolymers and/or acrylic acid/itaconic acid copolymers. It is self-evident that with the use of an extremely reactive glass powder a less reactive polycarboxylic acid will be employed in order to obtain a satisfactory hardening characteristic.

In order to accelerate and improve hardening of said glass ionomer cements chelating agents may be added during mixing, in a manner known from DE A 2.319.715. Instead of the customary use of the aqueous polycarboxylic acid solution as mixing fluid, the glass powder may also be pre-mixed in the corresponding ratio with the dry powdered polycarboxylic acid, as the solid substances do not undergo reaction with each other. In that case water is used as mixing fluid, preferably an aqueous solution of a chelating agent together with conventional additives such as bacteriostatic agents, if appropriate.

In order to avoid metering errors and to attain optimum mechanical properties, the powders may be used in pre-dosaged form. For example, the glass powder is metered out in plastic containers. Then the cement can either be mixed mechanically within said plastic capsules, or the container may be emptied and the mix prepared by hand. The aqueous polycarboxylic acid solution in such a case is metered, for example, with a dripping bottle or with a syringe. The use of the powder of the invention in so-called shaker capsules, e.g., corresponding to DE A 2.324.296, is suitable. A predetermined quantity of powder is held in readiness in a so-called main compartment, while the fluid is contained in a separate cushion beneath a lateral clip. By exerting pressure on said clip the fluid is sprayed through a bore into the main compartment and is then available for mechanical mixing. In both types of capsules the pure glass powder may be replaced by a mixture of glass powder and dry polycarboxylic acid in predetermined quantities. The fluid component is then water or an aqueous solution of a chelating agent.

The use of the mixture of glass powder and dry polycarboxylic acid is especially advantageous if said mixture is pelletised. To this end, the dry polycarboxylic acid is used in finely divided form after removal of coarse portions. After thorough blending of said polycarboxylic acid powder with the glass powder pellets may be made in a conventional pelletising machine. The compacting pressure must be selected such that after the addition of the mixing fluid (water or aqueous tartaric acid solution, for example), the pellets can still be readily worked into a cement while, on the other hand, they possess sufficient mechanical stability for transportation. Pellets made in this way permit especially simple mixing into a cement paste after brief dissolution, e.g., in the corresponding amount of tartaric acid solution. The mixing fluid may be added, for example, from a drip bottle or from a syringe.

According to the invention, the poly(dialkylsiloxane) may be linear or cyclic. It may further be a blend of different poly(dialkylsiloxane)s, e.g. a blend of a poly(dimethylsioxane) of high kinematic viscosity and a poly(dimethylsiloxane) of low kinematic viscosity. It is further preferred that the alkyl groups of the poly(dialkylsiloxane) are methyl groups. The kinematic viscosity is preferably in the range of about 1 cSt to about 100.000 cSt at 25° C. [about 1 to about 100.000 mm²/s], preferably about 100 cSt to about 10.000 cSt at 25° C. [about 100 to about 10.000 mm²/s], even more preferably about 500 cSt to about 5000 cSt at 25° C. [about 100 to about 10.000 mm²/s]. The best results are obtained with a viscosity of about 1000 cSt at 25° C. [about 1000 mm²/s].

According to the invention, the particles of the fluorosilicate glass powder have preferably an average size of about 0.5 μm to about 200 μm, more preferably about 3 μm to about 150 μm, even more preferably about 3 μm to about 100 μm and in particular about 20 μm to about 80 μm.

It is preferred that the aqueous acid solution comprises an inorganic acid or an organic acid. It is even more preferred that the aqueous acid solution comprises an organic acid, wherein the organic acid is preferably a polymer, e.g. a polyacrylic acid. According to the invention, the aqueous acid solution has a pH in the range of 2 to 7.

The present invention also relates to a process for the preparation of a self hardening glass carbomer composition. According to the process according to the invention, a fluorosilicate glass powder is treated with in subsequent order:

(a) a poly(dialkylsiloxane) having terminal hydroxyl groups, wherein the alkyl groups contain 1 to 4 carbon atoms,

(b) an aqueous acid solution,

(c) separating the treated fluorosilicate glass powder from the aqueous acid solution.

The present invention also relates to the use of the self hardening glass carbomer composition according to the invention as a (temporarily) dental filling material, a denting bonding cement and a bone cement. The self hardening glass carbomer composition according to the invention can also be used as bone replacing material in orthopaedic surgery, e.g. as implants or coating materials for joint cavities.

### EXAMPLES

### Example 1

The following compositions were prepared from the following ingredients:
(a) a polydimethylsiloxane having a kinematic vicsosity of 1000 cSt indicated by S20;
(b) a conventional fluorosilicate glass powder; and
(c) a conventional aqueous polyacrylate solution.

The fluorosilicate glass powder and aqueous polyacrylate solution used to prepare the compositions were taken from A3 APLICAP capsules from 3M ESPE. The amounts of the ingredients are listed in Table 1, wherein 5 wt. % additional fluorosilicate glass powder equals about 0.015 g fluorosilicate glass powder and wherein 0.0015 g S20 equals about 1.6% additional liquid added to the normal quantity of aqueous polyacrylate solution (about 0.0920 g).

TABLE 1

Composition relative to contents of
commercially available A3
APLICAP capsule

| | | Additional fluorosilicate |
|---|---|---|
| Product | S20 (g) | glass powder (wt. %) |
| 4P | 0.0015 | 5.00 |
| 5P | 0.0015 | 6.25 |
| 6P | 0.0015 | 7.50 |
| 8P | 0.0015 | 10.00 |
| 12P | 0.0045 | 15.00 |

### Example 2

The compositions according to Example 1 were evaluated in an in-vitro wear test in the ACTA-wear machine which is a three body wear system designed to simulate the wear that occurs in the oral cavity (cf. de Gee et al., 1994, 1996). Two reference materials (IFMC and KPFA; KPFA is KetacMolar® from 3M ESPE) were tested for comparison. In this test, two wheels (a first wheel containing the sample to be tested and a second wheel being an antagonist) rotate in different directions but with 15% difference in circumferential speed (called slip) while having near contact on the circumference. Test samples are situated on the circumference of the first wheel. The force with which the two wheels work against each other is adjusted 1t about 15 N. Both wheels are situated in slurry of rice flour and husks of while millet spray in a buffer solution. During the wear test, the food is pressed between the wheels creating a wear track into the test samples, leaving an untouched area at both sides of the reference for the determination of the wear. The material lost by wear was determined b evaluating 10 samples with a profilometer.

The samples were prepared in the first wheel (size approximately 10×15×3 mm). During setting, the compositions according to Example 1 were stored in a 100% relative humidity oven at 37° C. After being set, the samples were glued on the first wheel by using a cyanoacrylate glue. Thereafter, the sample wheel was wet ground until a uniform cylindrical outer surface was obtained. The wear grinding was performed in the wear testing machine with carburondrum and diamond wheels up to a grit of 1000. During this procedure, a layer of 100 μm was removed from the outer surface. Subsequently, the wear test was started which was conducted at 37° C. and pH 7.0. Wear data were obtained after 1 day, 4 days and 8 days. The data obtained are shown in Table 2, wherein a rating of below 60 is acceptable wherein lower data equals a higher hardness.

TABLE 2

| Time | | | | | | |
|---|---|---|---|---|---|---|
| (days) | IFMC | KPFA | 5P | 6P | 8P | 12P |
| 1 | 135.7 | 49.5 | 57.8 | 54.1 | 53.9 | 71.5 |
| 4 | 68.2 | 43.1 | 47.7 | 42.8 | 45.2 | 57.4 |
| 8 | 62.7 | 41.5 | 43.8 | 42.7 | 40.0 | 57.4 |

The data of Table 2 show that the hardness of the samples increase with time. From the data of Table 2, it is concluded that IFMC is inferior to all tested samples prepared from compositions according to the present invention. In addition, KPFA shows an infererior performance when compared with sample 8P according to the present invention.

### Example 3

In this example solubility tests were performed. The test was performed as follows. The weight of hardened samples having a diameter of about 0.4 to about 0.6 cm and having a thickness of about 1 to about 1.5 mm were was determined as reference. In the test, these samples were immersed in water at various pH values wherein the pH was adjusted with citric acid. A pH value of 2.5 was tested since it simulates the pH that can occur in between molars. The test was performed over a period of about 15 days. At several time intervals the weight of the test samples was determined wherein a greater weight loss indicated a higher solubility of the material. The data are expressed as % solubility (calculated on the original weight and the weight loss at the indicated periods) and are shown in Table 3.

TABLE 3

| | pH 2.5 | | pH 3.2 | | pH 7.0 | |
|---|---|---|---|---|---|---|
| Time (h) | KPFA | 5P | KPFA | 5P | KPFA | 5P |
| 0.0 | 0.0 | 0.0 | 0.0 | 0.0 | 0.0 | 0.0 |
| 1.5 | 12.1 | 11.7 | 7.1 | 5.0 | — | — |
| 72.0 | 20.2 | 16.9 | 10.9 | 8.0 | 2.6 | 1.1 |
| 144.0 | 25.2 | 19.2 | 19.9 | 14.2 | — | — |
| 360.0 | 27.4 | 21.6 | 19.9 | 16.3 | 5.5 | 1.8 |

From the data in Table 3 it appears that the sample prepared from the composition according to the invention showed an improved solubility performance than the commercially available material KPFA.

1. A self-hardening glass carbomer composition obtainable by treating a fluorosilicate glass powder with:
(a) a poly(dialkylsiloxane) having terminal hydroxyl groups, wherein the alkyl groups contain 1 to 4 carbon atoms,
(b) an aqueous acid solution,
(c) separating the treated fluorosilicate glass powder from the aqueous acid solution.

2. The self hardening glass carbomer composition according to claim 1, wherein the poly(dialkylsiloxane) is linear or cyclic.

3. The self hardening glass carbomer composition according to claim 1, wherein the alkyl groups of the poly(dialkylsiloxane) are methyl groups.

4. The self-hardening glass carbomer composition according to claim 1, wherein the poly(dialkylsiloxane) has a kinematic viscosity in the range of about 1 to about 100,000 cSt at 25° C.

5. The self-hardening glass carbomer composition according to claim 1, wherein the particles of the fluorosilicate glass powder have an average size of about 0.5 to about 200 μm.

6. The self hardening glass carbomer composition according to claim 1, wherein the aqueous acid solution comprises an inorganic acid or an organic acid.

7. The self hardening glass carbomer composition according to claim 6, wherein the organic acid is a polymer.

8. The self hardening glass carbomer composition according to claim 1, wherein the aqueous acid solution has a pH in the range of 2 to 7.

9. Process for the preparation of a self hardening glass carbomer composition, wherein a fluorosilicate glass powder is treated with:
(a) a poly(dialkylsiloxane) having terminal hydroxyl groups, wherein the alkyl groups contain 1 to 4 carbon atoms,
(b) an aqueous acid solution,
(c) separating the treated fluorosilicate glass powder from the aqueous acid solution.

10. (canceled)

11. A dental filling material prepared from the glass carbomer composition of claim 1.

12. A dental bonding cement prepared from the glass carbomer composition of claim 1.

13. A bone cement prepared from the glass carbomer composition of claim 1.

14. A bone replacement material prepared from the glass carbomer composition of claim 1.
